(19) United States
(12) Patent Application Publication
(10) Pub. No.: US 2025/0082563 A1
(43) Pub. Date: Mar. 13, 2025

(54) ANHYDROUS COMPOSITION FOR SKINCARE AND/OR ANTI-TRANSFER MAKEUP

(71) Applicant: L V M H RECHERCHE, Saint Jean De Braye (FR)

(72) Inventors: Valérie ALARD, Saint Jean De Braye (FR); Anne-Sophie RATTINA, Saint Jean De Braye (FR); Laurent NEAU, Saint Jean De Braye (FR); Ludivine LAURENT, Saint Jean De Braye (FR)

(73) Assignee: L V M H RECHERCHE, Saint Jean De Braye (FR)

(21) Appl. No.: 18/723,140

(22) PCT Filed: Dec. 19, 2022
(86) PCT No.: PCT/FR2022/052427

§ 371 Date: Jun. 21, 2024

(30) Foreign Application Priority Data
Dec. 21, 2021 (FR) 2114155

(51) Int. Cl. A61K 8/88; A61Q 1/06

(52) U.S. Cl. CPC A61K 8/88; A61Q 1/06; A61K 2800/31; A61K 2800/592

(57) ABSTRACT

The present invention relates to an anhydrous cosmetic composition which is preferably solid, comprising, in a physiologically acceptable medium, at least:-a polyamide-type lipophilic polycondensate,-a compound promoting the adhesion of the composition to keratin materials chosen in particular from the group consisting of: vinylpyrrolidone (VP) and alkene copolymers having from 2 to 20 carbon atoms, sugar esters, preferably sucrose acetate isobutyrate (SAIB), branched dextrin esters, and mixtures thereof,-in addition optionally at least one coloring material, characterised in that it has a total wax content of less than 7% by weight, preferably less than or equal to 5% by weight, or contains no wax(es).

### FIELD OF THE INVENTION

The present invention relates to the cosmetic field and in particular to anhydrous cosmetic compositions for the care and/or makeup of keratin materials, in particular of the skin and lips.

### STATE OF THE ART

There is a strong expectation among consumers for anhydrous skincare and makeup products with good staying power on keratin materials, in particular good staying power on the skin or lips, and in particular products which are resistant to all “conditions of life”, including so-called “extreme” conditions: high temperature and humidity, sweat and sebum (consequences of temperature) and widespread mask wearing (involving areas of friction and transfer).

The use of long-wearing polymers, in particular silicones, volatile oils, and waxes in particular in solid compositions is known from the prior art, but the staying effectiveness (no migration and/or non-transfer) and/or or sensoriality (comfort) on the skin are not always optimal, particularly in extreme conditions.

Consequently, there remains a need to develop new anhydrous products with improved staying power, without compromising on the desired cosmetic and sensory (comfort) properties. Advantageously, products having little or no volatile oils, and, for solid compositions, little or no waxes, are sought.

The Applicant has demonstrated that the combination of a polyamide-type lipophilic polycondensate and one or more compounds promoting the adhesion of the product to the skin and/or lips, allowed to improve the cohesion and adhesion properties of the product film deposited on the skin/lips and consequently its resistance to external stress. Surprisingly, this combination allows to limit the sticky feel of the polyamide-type lipophilic polycondensate alone, and not to damage the array formed by the polyamide-type lipophilic polycondensate in the fatty phase, thanks to a chemical interaction polyamide-type lipophilic polycondensate/adhesion agent. This combination allows to formulate anhydrous compositions, particularly solid ones, without silicone resin and with a wax content of less than 7% relative to the total weight of the composition.

### DISCLOSURE OF THE INVENTION

A first aspect of the invention is an anhydrous cosmetic composition, which is preferably solid, comprising, in a physiologically acceptable medium, at least:
a polyamide-type lipophilic polycondensate,
a compound promoting the adhesion of the composition to keratin materials chosen in particular from the group consisting of:
   Vinylpyrrolidone (VP) and alkene copolymers having from 2 to 20 carbon atoms,
   Sugar esters, preferably sucrose acetate isobutyrate (SAIB),
   branched dextrin esters,
   and mixtures thereof, and
in addition optionally at least one coloring material, characterized in that it has a total wax content of less than 7% by weight, preferably less than or equal to 5% by weight, or contains no wax(es).

‘Keratin materials’ mean in particular the skin and/or the lips. The invention also relates to a cosmetic method for the care and/or makeup of keratin materials, in particular the skin and/or lips, comprising the application, to said keratin materials, of a composition according to the invention.

### DETAILED DESCRIPTION OF THE INVENTION

Thus, a first aspect of the invention is therefore an anhydrous cosmetic composition, which is preferably solid, comprising, in a physiologically acceptable medium, at least:
a polyamide-type lipophilic polycondensate,
a compound promoting the adhesion of the composition to keratin materials chosen in particular from the group consisting of:
   vinylpyrrolidone (VP) and alkene copolymers having from 2 to 20 carbon atoms,
   Sugar esters, preferably sucrose acetate isobutyrate (SAIB),
   branched dextrin esters,
   and mixtures thereof, and
in addition optionally at least one coloring material,
characterized in that it has a total wax content of less than 7% by weight, preferably less than or equal to 5% by weight, or contains no wax(es).

The composition of the invention is an anhydrous composition.

“Anhydrous” means in particular that water is preferably not deliberately added to the compositions but may be present in trace amounts in the various compounds used in the compositions. In particular, the composition according to the invention comprises less than 4% by weight of water, preferably less than 3%, preferably less than 2%, more preferably less than 1%, even more preferably less than 0.5%. by weight of water, relative to the total weight of said composition, or even is completely free of water.

The composition of the invention can be liquid or solid.

According to a first embodiment, the composition is liquid.

“Liquid” according to the invention means a fluid composition which is capable of flowing under its own weight, at 20° C. and at atmospheric pressure (760 mm Hg), as opposed to compositions called solid compositions.

According to another embodiment, the composition is solid, as defined by viscoelastic parameters according to the following protocol for example.

The viscoelastic parameters of the products (compositions of the invention) are measured using a DHR2 rheometer from TA Instruments, at 25° C. and at atmospheric pressure (760 mm Hg). A pellet of product is cut out and deposited between 2 smooth metal planes of 20 mm in diameter and 1 mm of air gap. The conservation modulus or elastic modulus G′ (Pa) and the delta phase angle) (° are recorded as a function of an increasing stress ranging from 0.1 Pa to 10000 Pa. The retained values are extracted from the linear domain in which the moduli are constant, that is to say at deformations less than 100 Pa. A product whose conservation modulus G′ is greater than or equal to 5000 Pa, and whose delta phase angle will be less than or equal to 30° will be considered as solid.

Thus, according to a particular and preferred embodiment, the composition of the invention is characterized by a conservation modulus G′ greater than or equal to 5000 Pa, and whose delta phase angle is less than or equal to 30°, as measured according to the protocol described above.

### Polyamide-Type Gelling Polymer (Promotes Film Cohesion)

The composition according to the invention comprises at least one polyamide-type lipophilic polycondensate, as fatty phase gelling polymer.

Polyamide-type polycondensates can form a three-dimensional network through hydrogen bond-type interactions, in which lipophilic oils have an affinity for acidic dimer chains. Applied to the surface of the skin and/or lips, they form a very structured network resistant to external stress (friction, contact, etc.) and which reforms quickly after shearing, while remaining comfortable. Their presence in the fatty phase thus gives the composition of the invention significantly improved staying power when it is applied in the form of a film to the surface of the skin and/or lips,

For the purposes of the invention, the term “polycondensate” means a polymer obtained by polycondensation, namely by chemical reaction between monomers having different functional groups chosen in particular from acid, alcohol and amine functions.

For the purposes of the invention, the term “polymer” means a compound having at least 2 repeating units, preferably at least 3 repeating units and better still 10 repeating units.

In the rest of the description ‘polyamide polymer’ or ‘polyamide polycondensate’ or ‘lipophilic polyamide polycondensate’ or ‘polyamide-type lipophilic polycondensate’ or ‘lipophilic gelling polymer of polyamide type’ are considered indifferently.

The polyamide-type polycondensate usable as fatty phase gelling agents in the composition of the invention is preferably chosen from ester-terminated polyamides, tertiary amide-terminated polyamides or polyalkyleneoxy-terminated polyamides or else ester-terminated poly(ester-amides).

In the context of the invention, the composition according to the invention advantageously comprises at least one ester-terminated polyamide and/or one ester-terminated poly(ester-amides).

According to a first preferred embodiment of the invention, the composition comprises at least one ester-terminated polyamide obtained by condensation of a C36 diacid, more particularly a linoleic acid dimer (also called dilinoleic acid), on ethylene diamine, the remaining acid endings being esterified with cetyl alcohol (C16), stearyl alcohol (C18) or their mixture. The copolymer thus obtained has the INCI name: ETHYLENEDIAMINE/STEARYL DIMER DILINOLEATE COPOLYMER. It is in particular sold by the company ARIZONA CHEMICAL under the trade names UNICLEAR 80 and UNICLEAR 100 VG respectively in the form of a gel at 80% (in active material) in a mineral oil and at 100% (in active material).

These mixtures are also sold by the company CRODA under the trade name OLEOCRAFT LP-10-PA-(MV) respectively at 99.7% (in active material) with a preservative. They have a softening point of 88° C. to 94° C. Its weight average molecular mass is preferably 6000, even more preferably 4000.

According to a second particularly preferred embodiment of the invention, the composition comprises at least one poly (ester-amide) with ester termination obtained by condensation of a C36 diacid, more particularly a dimer of hydrogenated linoleic acid (also called hydrogenated dilinoleic acid) and neopentyl glycol, on ethylene diamine; the remaining acid endings being esterified with stearyl alcohol (C18).

The copolymer thus obtained is an ethylenediamine bis-stearyl ethylenediamine/neopentyl glycol/stearyl dibenzoate dimer copolymer, with the INCI name: POLYAMIDE-8.

The copolymer with the INCI name POLYAMIDE-8 is for example marketed under the name OLEOCRAFT LP-20 by the company CRODA.

In general, the polymers as described below are in the form of mixtures of polymers, these mixtures may also contain a synthesis product corresponding to a diester.

A mixture of lipophilic gelling agents among those described above is for example a mixture of ester-terminated poly (ester-amides) and polyalkyleneoxy-terminated polyamides whose molecular weights are approximately between 4500 and 30000 Daltons (Da).

According to a particular embodiment, the polyamide-type lipophilic polycondensate used according to the invention is present in the composition in an amount of active material ranging from 5 to 30% by weight relative to the total weight of the composition.

According to a particular embodiment, in particular in the case of a liquid composition according to the invention, the polyamide-type lipophilic polycondensate used according to the invention is present in the composition in an amount of active material ranging from 5 to 20% by weight, in particular from 5 to 15% by weight relative to the total weight of the composition.

According to another particular embodiment, in particular in the case of a solid composition according to the invention, the polyamide-type lipophilic polycondensate used according to the invention is present in the composition in an amount of active material ranging from 15% to 30% by weight, preferably from 20% to 25% by weight relative to the total weight of the composition.

According to a particular embodiment of the invention, the composition does not comprise a lipophilic co-gelling agent promoting the cohesion of the film on the skin. In particular, it does not contain silicone resin.

### Compound Promoting the Adhesion of the Film on the Skin and/or Lips

The composition of the invention comprises one or more compounds having good adhesion properties on the skin and/or lips, dispersible in the fatty phase, polymeric or non-polymeric, and distinct from the lipophilic polyamide polycondensate described above.

This compound promoting the adhesion of the composition to keratin materials (otherwise called ‘adhesive compound’) according to the invention is capable of guaranteeing optimal adhesion to the skin and/or lips throughout the day.

According to a particular embodiment, this compound promoting the adhesion of the film to the skin and/or lips is characterized by an adhesion work measurement, as defined by the following protocol.

The adhesion work of a raw material (for example: a polymer) is measured at room temperature (22° C.+/−2° C.) and at atmospheric pressure (760 mm Hg) using the method called “Delrin finger” method.” The apparatus used is a TAXT Plus texturometer from Stable Micro Systems. The method consists of inserting a polyoxymethylene (Delrin) cylinder of 12.7 mm in diameter, which is rounded at its end, into a pot 6 cm in diameter and 4 cm in height, filled with the raw material (for example: polymer) to be tested. The insertion depth is 13 mm, at a speed of 2 mm/sec. At the end of this insertion, the cylinder is withdrawn at a speed of 2 mm/sec.

The force of resistance to removal of the cylinder is measured until the polymer stuck to the cylinder is completely released, corresponding to a return to a zero measured force. The calculated adhesion work, expressed in N/mm, corresponds to the total area under the abscissa axis.

Thus, in the context of the invention, use will preferably be made of a compound which promotes adhesion of the film to the skin and/or lips having an adhesion work greater than or equal to 2 N/mm, preferably greater than or equal to at 4 N/mm, and ranging in particular from 5 N/mm to 35N/mm, or even from 7 N/mm to 30N/mm, measured for example by the protocol described above.

Mention can in particular be made of the following adhesion polymers, having an adhesion work measured according to the protocol described above:
UNIMER U1946 (VP/hexadecene copolymer, INCI name: VP/hexadecene and octyldodecanol): 7.7 N/mm
CGT-UNIFILMA HVY (85%) (dextrin isostearate): 20.7 N/mm
EASTMAN SUSTANE SAIB (sucrose acetate isobutyrate): 24.2 N/mm.

Thus, according to a particular embodiment of the invention, said compound promoting the adhesion of the film to keratin materials is chosen from the group consisting of:
vinylpyrrolidone (VP) and alkene copolymers having from 2 to 20 carbon atoms,
Sugar esters, preferably sucrose acetate isobutyrate (SAIB),
branched dextrin esters,
and mixtures thereof.

### Vinylpyrrolidone (VP) and Alkene Copolymers

According to a first embodiment, the composition of the invention comprises a polymer chosen from the vinylpyrrolidone (VP) and alkene copolymers having from 2 to 20 carbon atoms, such as the copolymers of VP/eicosene, VP/hexadecene, VP/styrene.

According to a particular and preferred embodiment, the composition of the invention comprises a VP/hexadecene copolymer, such as that of the INCI name: VP/hexadecene and octyldodecanol, marketed under the name UNIMER U-1946.

The vinylpyrrolidone (VP) and alkene copolymer(s) having from 2 to 20 carbon atoms, in particular the VP/hexadecene copolymer, is (are) present in the composition in a total content ranging from 2 to 20%, in particular from 5 to 15%, by weight relative to the total weight of the composition.

### Sugar Esters

According to another particular embodiment, the composition of the invention comprises a compound promoting the cohesion of the film chosen from sugar esters, in particular sucrose acetate isobutyrate marketed under the name EASTMAN SUSTANE SAIB by the company EASTMAN. This compound can be prepared by known techniques of reaction between a sucrose and acetic and isobutyric anhydrides, followed by extensive purification by vacuum distillation. The degree of esterification is almost complete (degree of substitution greater than 7.5 for a maximum degree of substitution of 8), with an acetate: isobutyrate ester ratio of 2:6. Patent U.S. Pat. No. 3,096,324 gives an example of preparation of SAIB.

The sucrose ester(s), in particular sucrose acetate isobutyrate, is (are) present in the composition in a total content ranging from 2 to 20%, in particular from 5 to 15%, by weight relative to the total weight of the composition.

### Branched Dextrin Esters

According to another particular embodiment, the composition of the invention comprises a compound promoting the adhesion of the film to keratin materials chosen from branched dextrin esters.

The branched dextrin ester according to the invention may comprise one esterified chain or several esterified chains, which are identical or different. It may for example be a dextrin esterified with several identical or different fatty acids. These dextrin esters can be prepared using conventional esterification methods.

According to a particular embodiment, the branched dextrin ester is a dextrin ester, for which the dextrin has an average degree of glucose polymerization of 3 to 150, and for which the fatty acid(s) comprise from 50% to 100% in moles, based on the total amount of fatty acid(s), one or more saturated branched fatty acids having 4 to 26 carbon atoms, and 0% to less than 50% in moles, based of the total amount of fatty acid(s), of one or more other fatty acids chosen from the group consisting of saturated linear fatty acids having from 2 to 22 carbon atoms, unsaturated linear or branched fatty acids, having 6 to 30 carbon atoms, and cyclic, saturated or unsaturated fatty acids having 6 to 30 carbon atoms, and for which the degree of substitution of dextrin by the fatty acid(s) is 1.0 to 3.0 per unit of glucose.

Such dextrin esters are described in particular in European application EP 2537865.

According to a preferred embodiment, the branched dextrin ester is chosen from dextrin isostearate, dextrin isoarachidate, dextrin isopalmitate, dextrin isononanoate, as well as mixtures thereof. Preferably, the branched dextrin ester is dextrin isostearate.

Among the compounds marketed, mention can be made of the dextrin isostearate UNIFILMA HVY marketed by the company Chiba Flour Milling Co.

According to one embodiment, the branched dextrin ester is present in the composition in a content ranging from 2 to 20%, in particular from 5 to 15%, by weight relative to the total weight of the composition.

According to a particular embodiment, the composition of the invention comprises at least one sucrose acetate isobutyrate such as that marketed under the name EASTMAN SUSTANE SAIB by the company EASTMAN.

According to another particular embodiment, the composition of the invention comprises at least one branched dextrin ester.

According to another particular embodiment, the composition of the invention comprises at least one sugar ester and a vinylpyrrolidone (VP) and alkene copolymer.

Thus, according to a particular embodiment, the composition of the invention comprises:
a polyamide-type lipophilic polycondensate with the INCI name POLYAMIDE-8, preferably in a content ranging from 5 to 30% by weight, in particular from 15% to 30% by weight relative to the total weight of the composition,
a compound promoting the adhesion of the composition on keratin materials chosen from the group consisting of:
   vinylpyrrolidone (VP) and alkene copolymers having from 2 to 20 carbon atoms,
   Sugar esters, preferably sucrose acetate isobutyrate (SAIB),
   and their mixture,
   and in addition optionally a coloring material

According to a particular embodiment, the composition of the invention comprises:
a polyamide-type lipophilic polycondensate, in particular with an active material content ranging from 5 to 30% by weight, preferably from 15 to 30%, in particular from 20 to 25% by weight relative to the total weight of the composition and
a VP/hexadecene copolymer, in particular in a content ranging from 2 to 20%, in particular from 5 to 15%, by weight relative to the total weight of the composition.

Preferably, in this particular embodiment, the polyamide-type lipophilic polycondensate, with the INCI name POLYAMIDE-8, is used.

According to another particular embodiment, the composition of the invention comprises:
a polyamide-type lipophilic polycondensate, in particular in a content ranging from 5 to 30% by weight, preferably from 15 to 30%, in particular from 20 to 25% by weight relative to the total weight of the composition and
a sucrose acetate isobutyrate, in particular in a content ranging from 2 to 20%, in particular from 5 to 15% by weight relative to the total weight of the composition.

Preferably, in this particular embodiment, the polyamide-type lipophilic polycondensate, with the INCI name POLYAMIDE-8, is used.

According to a particular embodiment, the composition of the invention comprises:
a polyamide-type lipophilic polycondensate, in particular in a content ranging from 5 to 30% by weight, preferably from 15 to 30%, in particular from 20 to 25% by weight relative to the total weight of the composition and
a VP/hexadecene copolymer, in particular in a content ranging from 2 to 20%, in particular from 5 to 15% by weight relative to the total weight of the composition, and
a sucrose acetate isobutyrate, in particular in a content ranging from 2 to 20%, in particular from 5 to 15% by weight relative to the total weight of the composition.

Preferably, in this particular embodiment, the polyamide-type lipophilic polycondensate, with the INCI name POLYAMIDE-8, is used.

### Oily Phase

The cosmetic composition of the invention comprises a fatty or oily phase.

“Oily phase” means an oil or a mixture of oils that are miscible with each other.

“Oil” means, within the meaning of the invention, a fatty substance, not soluble in water, liquid at 25° C. and atmospheric pressure.

The composition of the invention comprises a wax content of less than 7% by weight, in particular less than or equal to 5%, preferably less than or equal to 2%, or contains no waxes.

In the case where the composition of the invention comprises one or more waxes in a content of less than 7% by weight of the composition, said waxes are chosen from waxes of natural origin, in particular waxes of plant origin.

Waxes of natural origin include beeswax, carnauba wax and candelilla wax.

According to a particular embodiment, the composition of the invention comprises less than 5% of silicone oils, in particular less than 2% of silicone oils, or contains no silicone oils.

Preferably, the composition of the invention comprises less than 5% by weight of volatile oils, in particular less than 2% by weight of volatile oils, or contains no volatile oils.

Thus, preferably, the composition of the invention does not comprise wax, silicone oil or volatile oil.

The oily phase of the composition will comprise at least non-volatile hydrocarbon oils, chosen from hydrocarbon oils of plant origin, synthetic C₁₀-C₄₀ ethers, synthetic C₁₀-C₄₀ esters, C₁₂-C₂₆ fatty alcohols, higher C₁₂-C₂₂ fatty acids, and mixtures thereof.

According to a particular embodiment, the composition will comprise one or more hydrocarbon oil(s), preferably chosen from ester oils.

Among the “ester oils”, mention can in particular be made of:
fatty acid esters, in particular of 4 to 22 carbon atoms,
synthetic esters such as oils of formula R₁COOR₂ in which R₁ represents the remainder of a linear or branched fatty acid including from 4 to 40 carbon atoms and R₂ represents a hydrocarbon chain which is in particular branched containing from 4 to 40 carbon atoms provided that R₁+R₂ is ≥16, such as isononyl isononanoate, 2-hexyl ethyl palmitate, octyledodecyl neopentanoate, 2-octyl dodecyl stearate, isostearyl isostearate, 2-octyl dodecyl benzoate, isopropyl myristate, isopropyl palmitate, butyl stearate, hexyl laurate, 2-ethyl-hexyl palmitate, 2-hexyl-decyl laurate, 2-octyl-decyl palmitate, 2-octyldodecyl myristate, 2-diethyl-hexyl succinate;
esters of linear fatty acids having a total carbon number ranging from 35 to 70;
hydroxylated esters, preferably having a total carbon number ranging from 35 to 70, such as polyglycerol-2 triisostearate, isostearyl lactate, octyldodecyl hydroxystearate, diisostearyl malate, glycerin stearate; diethylene glycol diisononanoate;
esters of aromatic acids and alcohols comprising 4 to 22 atoms;
esters of fatty alcohol or C24-C28 branched fatty acids;
polyesters resulting from the esterification of at least one triglyceride of hydroxylated carboxylic acid(s) with an aliphatic monocarboxylic acid and with an aliphatic dicarboxylic acid, which is optionally unsaturated; and mixtures thereof.

According to a particular embodiment, the composition of the invention will comprise at least one ester oil chosen from the group consisting of synthetic esters, hydroxylated esters and mixtures thereof.

According to a particular and preferred embodiment, the composition comprises at least one hydroxylated ester.

According to a particular embodiment, the composition comprises at least two ester oils from distinct families, in particular an oil from the synthetic ester family and an oil from the hydroxylated ester family. The total amount of oils present in the composition according to the invention will generally range from 35 to 65%, preferably from 40 to 60% by weight relative to the total weight of the composition.

According to a particular embodiment, the composition comprises one or more ester oils as defined above in a total content ranging from 35 to 65%, preferably from 40 to 60% by weight relative to the total weight of the composition.

The oily phase may also comprise a vegetable resin, in particular chosen from the group consisting of a candelilla resin, a carnauba resin, and a rosinate.

According to a particular embodiment, a candelilla resin will be used. Mention can in particular be made of the candelilla resin marketed under the name CANDELILLA RESIN E2 by the company Japan natural products Co., Ltd.

### Coloring Materials

According to a particular embodiment of the invention, in particular for tinted care compositions or makeup compositions, the composition comprises at least one coloring material.

Coloring material within the meaning of the present invention means a compound capable of producing a colored optical effect when formulated in sufficient amount in an appropriate cosmetic medium.

A coloring material can be chosen from organic or inorganic coloring materials, materials with an optical effect, and mixtures thereof, soluble or insoluble in the oily phase of the invention.

According to a particular embodiment, the coloring material(s) are chosen in particular from mineral pigments, organic pigments, and mixtures thereof.

“Pigments” mean white or colored, mineral or organic particles, which are insoluble in an aqueous solution, intended to color and/or opacify the resulting deposit. Mineral pigments, organic pigments, and composite pigments (that is to say pigments based on mineral and/or organic materials) can be mentioned.

Among the “mineral pigments”, mention can be made, as examples, of titanium dioxide (rutile or anatase), possibly surface treated; black, yellow, red and brown iron oxides; manganese violet; ultramarine blue, chromium oxide, hydrated chromium oxide and ferric blue.

For compositions intended for the lips, titanium dioxide may be mentioned as examples; black, yellow, red and brown iron oxides and manganese violet.

Among the “organic pigments”, mention can be made, for example, of the pigments D & C red no 19; D & C red no 9; D&C Red no 22; D&C Red no 21; D&C Red no 28; D & C Yellow no 6; D&C Orange no 4; D&C Orange no 5; D&C Red no 27; D & C red no 13; D&C Red no 7; D&C Red no 6; D & C Yellow no 5; D & C Red no 36; D&C Red no 33; D&C Orange no 10; D & C yellow no 6; D&C Red no 30; D & C red no 3; D&C Blue 1; carbon black and lacquers.

In particular, the coloring material(s) are present in the composition in a content ranging from 2% to 20% by weight, preferably from 3% to 15% by weight relative to the total weight of the composition.

According to a particular method, the total content of coloring matter will range from 2 to 10% by weight relative to the weight of the composition.

### Other Additional Ingredients

The composition of the invention may further comprise at least one additional ingredient chosen from antioxidants, perfumes, preservatives, cosmetic active ingredients, fillers, and mixtures thereof.

According to a particular embodiment, the composition does not comprise any filler or a content less than or equal to 5% by weight, in particular less than 2% by weight, or even less than 1% by weight relative to the total weight of the composition.

According to a particular embodiment, the composition further comprises one or more UV filters. The UV filter(s) may be chosen from inorganic UV filters, organic UV filters and mixtures thereof.

“Inorganic UV filters” are generally metal oxide pigments chosen from titanium, zinc, iron, zirconium, cerium oxides or mixtures thereof.

“Inorganic UV filters” can be water-soluble or fat-soluble.

According to a particular embodiment, a solar composition of the invention comprises an inorganic UV filter chosen from a titanium oxide, a zinc oxide and their mixture, and a liposoluble organic UV filter.

### GALENIC

The composition of the invention is an anhydrous composition.

The composition can be liquid or solid, preferably solid.

It may be a liquid composition for the lips, a lip stick, a fluid foundation, a stick foundation, a complexion corrector or concealer, an eyelid product, an eyebrow product or else an eyeliner.

According to a first embodiment, the composition is an anhydrous skin and/or lip care composition.

According to another embodiment, the composition is an anhydrous makeup composition for the skin and/or lips, in particular a liquid composition for the lips, a fluid foundation, a foundation stick, a complexion corrector or concealer, an eyelid product, an eyebrow product, an eyeliner or a lip stick (lip color stick).

According to another embodiment, the composition is a sunscreen product.

According to a particular embodiment, the composition is a liquid composition for care and/or makeup for the skin and/or lips, in particular a fluid foundation for the skin or a liquid composition for the lips.

According to another particular embodiment of the invention, the composition is a solid composition, in the form of a stick for the care and/or makeup of the skin and/or lips, in particular a stick for the skin (for example: stick foundation, complexion corrector, eyelid stick, eyebrow pencil, eye pencil) or a lip stick (for example lip color stick).

Preferably, it will be a lip color stick or a stick foundation.

According to a particular embodiment, the composition is a liquid anhydrous composition comprising:
a polyamide-type lipophilic polycondensate, in particular in a content ranging from 5 to 30% by weight, preferably 5 to 20% and better still from 5 to 15% by weight relative to the total weight of the composition,
a compound chosen from the group consisting of:
   vinylpyrrolidone (VP) and alkene copolymers having from 2 to 20 carbon atoms,
   Sugar esters, preferably sucrose acetate isobutyrate (SAIB),
   and their mixture,
and pigments, characterized in that it comprises one or more ester oils in a total content ranging from 35 to 65% by weight relative to the total weight of the composition.

According to a particular embodiment, the composition is a liquid anhydrous composition comprising:
a polyamide-type lipophilic polycondensate, in particular in a content ranging from 5 to 30% by weight, preferably 5 to 20% and better still from 5 to 15% by weight relative to the total weight of the composition,
a sucrose acetate isobutyrate (SAIB), optionally in association with a vinylpyrrolidone (VP) and alkene copolymer having from 2 to 20 carbon atoms,
and pigments.

According to another particular embodiment, the composition is a solid anhydrous composition comprising:
a polyamide-type lipophilic polycondensate, in particular in a content ranging from 15 to 30% by weight relative to the total weight of the composition,
a sugar ester, preferably sucrose acetate isobutyrate (SAIB), or a dextrin ester, preferably sucrose acetate isobutyrate (SAIB),
a vinylpyrrolidone (VP) and alkene copolymer having from 2 to 20 carbon atoms,
and pigments.

According to another particular embodiment, the composition is a solid anhydrous composition comprising:
a polyamide-type lipophilic polycondensate, in particular in a content ranging from 15 to 30% by weight relative to the total weight of the composition,
a compound chosen from the group consisting of:
   esters, preferably sucrose acetate isobutyrate (SAIB), and dextrin esters,
   preferably sucrose acetate isobutyrate (SAIB).

According to a particular embodiment, the composition is a solid anhydrous composition comprising:
a polyamide-type lipophilic polycondensate in a content ranging from 15 to 30% by weight relative to the total weight of the composition,
a compound chosen from the group consisting of:
   vinylpyrrolidone (VP) and alkene copolymers having from 2 to 20 carbon atoms,
   Sugar esters, preferably sucrose acetate isobutyrate (SAIB),
   and their mixture,
and pigments.

According to a particular embodiment, the composition is a solid anhydrous composition comprising:
a polyamide-type lipophilic polycondensate, in particular in a content ranging from 15 to 30% by weight relative to the total weight of the composition,
a compound chosen from the group consisting of:
   vinylpyrrolidone (VP) and alkene copolymers having from 2 to 20 carbon atoms,
   Sugar esters, preferably sucrose acetate isobutyrate (SAIB),
   and their mixture,
and pigments,
   characterized in that it comprises one or more ester oils in a total content ranging from 35 to 65% by weight relative to the total weight of the composition.

According to a particular embodiment, the composition is a solid anhydrous composition comprising:
a polyamide-type lipophilic polycondensate, in particular in a content ranging from 15 to 30% by weight relative to the total weight of the composition,
a sucrose acetate isobutyrate (SAIB), optionally in association with a vinylpyrrolidone (VP) and alkene copolymer having from 2 to 20 carbon atoms,
and pigments.

### COSMETIC METHOD

The invention also relates to a cosmetic method for the care and/or makeup of keratin materials comprising the application, to said keratin materials, in particular the skin and/or the lips, preferably the lips, of an anhydrous composition according to the invention.

In particular, the anhydrous composition of the invention comprises, in a physiologically acceptable medium:
a polyamide-type lipophilic polycondensate, in particular in a content ranging from 5 to 30% by weight, preferably from 15 to 30% by weight relative to the total weight of the composition,
a compound chosen from the group consisting of:
   vinylpyrrolidone (VP) and alkene copolymers having from 2 to 20 carbon atoms,
   Sugar esters, preferably sucrose acetate isobutyrate (SAIB),
   and their mixture,
and pigments.

Preferably, the polyamide-type lipophilic polycondensate, INCI name POLYAMIDE-8, is used.

The particular embodiments described above illustrate makeup compositions which can be used in the cosmetic method according to the invention.

According to a particular embodiment, the composition applied to the skin is a skin care and/or makeup composition, in particular a fluid or stick foundation.

According to another particular embodiment, the composition applied to the lips is a liquid or solid lip makeup composition, preferably a solid lip makeup composition, in particular a lip stick.

The method according to the invention is in particular a method for caring for and/or making up the skin and/or lips, intended to deposit a film of non-transfer composition on the skin and/or lips.

The invention will now be illustrated in the following non-limiting examples. The % are expressed as % by weight relative to the total weight of the composition, unless otherwise indicated.

### EXAMPLES

### Example 1: Negative Effect of the Addition of Waxes (Carnauba Waxes and Respectively Microcrystalline Wax) on the Staying Power of the Shine and the Film and/or the Non-Transfer Aspect of an Anhydrous Lip Color Stick Composition According to the Invention

An anhydrous stick composition was formulated according to the invention, and comparative compositions in which contents (7% and 10%) were added respectively of Carnauba wax (vegetable wax) and of microcrystalline wax (synthetic wax).

| | | Comparison | Comparison | | |
|---|---|---|---|---|---|
| | | 1 | 2 | Comparison 3 | Comparison 4 |
| | | 7% | 10% | 7% | 10% |
| Ingredients (INCI | | carnauba | carnauba | microcrystalline | microcrystalline |
| name) | Invention | wax | wax | wax | wax |
| POLYAMIDE-8 | 25 | | | | |
| (SP OLEOCRAFT | | | | | |
| LP-20 MBAL-PA- | | | | | |
| (MV) from CRODA) | | | | | |
| VP/HEXADECENE | 5 | | | | |
| COPOLYMER | | | | | |
| (UNIMER U-1946) | | | | | |
| SUCROSE ACETATE | 5 | | | | |
| ISOBUTYRATE | | | | | |
| (SAIB 100) | | | | | |
| POLYGLYCERYL-10 | 20 | | | | |
| PENTAISOSTEARATE | | | | | |
| DIISOSTEARYL | 20 | | | | |
| MALATE | | | | | |
| PIGMENTS | 5 | | | | |
| CERA CARNAUBA | — | 7 | 10 | 7 | |
| (CARNAUBA WAX T- | | | | | 10 |
| 1 INA TRADING) | | | | | |

| | |
|---|---|
| POLYGLYCERYL-2 | qsp 100 |
| TRIISOSTEARATE | |

The formulas were prepared according to the following method:

The ingredients of the continuous oil phase (including waxes) are weighed into the main beaker.

The mixture is then heated to 90° C. in a water bath and dispersed with a Rayneri until a homogeneous transparent phase is obtained.

The pigments are weighed into a capsule, added to the main beaker and mixed for 15 minutes.

The composition obtained is solid.

The formulas were then evaluated on the one hand for the staying power of the film and the shine of the composition of the invention and on the other hand on the staying power (non-transfer) aspect.

### Film and Shine Staying Power

The formulas were therefore evaluated by a panel of experts, for the staying power of the film and the shine of the composition of the invention, according to the following protocol:
Each formula is applied in half-lips, the formula of the invention on the left part and the comparative formula on the right part;
Photos of the lips on which the formulas were applied were taken immediately after application, at T+2 hours and at T+4 hours after meals;
The experts evaluated the effect of the addition of waxes on the staying power of the film and the shine of the comparative compositions compared to the formula of the invention (control) and assigned the following notes:
   +: significant improvement in the staying power of the comparative formula compared to the formula of the invention
   −: significant deterioration in the staying power of the comparative formula compared to the formula of the invention
   0: no impact on the staying power of the formula (same as control)

The results of the evaluations are presented in the following tables: the same results were observed for carnauba wax and microcrystalline wax.

7% carnauba wax (Comparison 1) or 7%
microcrystalline wax (Comparison 3)

| | Staying | | | Staying |
|---|---|---|---|---|
| | power of | Staying power of the | | power of |
| | the film | film (+4 hours after | Immediate | the shine |
| | (+2 h) | meal) | shine | (+2 h) |
| Panelist 1 | 0 | 0 | 0 | 0 |
| Panelist 2 | 0 | 0 | 0 | 0 |

No impact on the staying power and shine with 7% waxes (carnauba or microcrystalline)

10% carnauba wax (Comparison 2) or 10%
microcrystalline wax (Comparison 4)

| | Staying | | | Staying |
|---|---|---|---|---|
| | power of | Staying power of the | | power of |
| | the film | film (+4 hours after | Immediate | the shine |
| | (+2 h) | meal) | shine | (+2 h) |
| Panelist 1 | - | -- | -- | - |
| Panelist 2 | - | -- | -- | - |

Negative impact on the staying power of the film and immediate shine. Adds stickiness

These results show that the addition of 10% by weight of Carnauba wax or microcrystalline wax relative to the total weight of the composition has a negative impact on the strength of the film and the shine of the composition of the invention.

### Staying Power Aspect (Non-Transfer)

At the same time, the formulas were also evaluated on the staying power aspect (non-transfer) according to the following protocol (“kiss” test):

It consists of making up the lips with the compositions (invention and comparisons in half-lips) and, after 15 minutes, kissing a sheet of paper.

It is then possible to evaluate the transfer of each composition (invention and comparisons) onto paper.

The transfer of each formula is assessed by visual observation and ratings are assigned thereto.
0: no transfer
−: light transfer
−−: average transfer
−−−: large transfer

The results are presented in the following table 4:

| | Transfer |
|---|---|
| Formulas tested | Assessment |
| Invention | - |
| Comparison 1 (7% carnauba) | -- |
| Comparison 2 (10% carnauba) | -- |
| Comparison 3 (7% microcrystalline) | -- |
| Comparison 4 (10% microcrystalline) | -- |

These results show that the addition of Carnauba wax or microcrystalline wax (at 7% or 10% by weight in the composition) reduces the staying power (non-transfer) property of the composition.

Consequently, on the basis of all these results, for good performance in film staying power and shine on the one hand and good non-transfer properties, use will be made of few, if any, waxes in the composition of the invention, and in any case a total content of less than 7% by weight of waxes so as not to alter the desired non-transfer properties.

### Example 2: Formulations

2-1 Non-transfer lip color stick

| | % by |
|---|---|
| Ingredients (INCI name) | weight |
| POLYAMIDE-8 | 24 |
| (SP OLEOCRAFT LP-20 MBAL-PA-(MV) from CRODA) | |
| SUCROSE ACETATE ISOBUTYRATE | 5 |
| (SAIB 100) | |
| CANDELILLA RESIN (CANDELILLA RESIN E2) | 5 |
| POLYGLYCERYL-10 PENTAISOSTEARATE | 21 |
| DIISOSTEARYL MALATE | 20 |
| PIGMENT | 8 |
| POLYGLYCERYL-2 TRIISOSTEARATE | Qsp100 |

The composition is prepared as described in Example 1.

Applied to the lips, the composition deposits a comfortable film, with very good staying power of the film and shine, and no transfer.

2-2 Non-transfer lip stick

| | % by |
|---|---|
| Ingredients (INCI name) | weight |
| POLYAMIDE-8 | 26 |
| (SP OLEOCRAFT LP-20 MBAL-PA-(MV) from CRODA) | |
| VP/HEXADECENE COPOLYMER | 9 |
| (UNIMER U-1946) | |
| POLYGLYCERYL-10 PENTAISOSTEARATE | 20 |
| DIISOSTEARYL MALATE | 20 |
| PIGMENT | 6 |
| POLYGLYCERYL-2 TRIISOSTEARATE | Qsp100 |

In an alternative example, the VP/HEXADECENE COPOLYMER (UNIMER U-1946) is replaced by an equivalent content of SUCROSE ACETATE ISOBUTYRATE (SAIB 100).

The composition is prepared as described in Example 1.

Applied to the lips, the composition deposits a comfortable film, with very good staying power of the film and shine, and no transfer.

1-11. (canceled)

12. An anhydrous cosmetic composition, which is preferably solid, comprising, in a physiologically acceptable medium, at least:
a polyamide-type lipophilic polycondensate,
a compound promoting the adhesion of the composition to keratin materials chosen in particular from the group consisting of:
vinylpyrrolidone (VP) and alkene copolymers having from 2 to 20 carbon atoms,
sugar esters,
branched dextrin esters,
and mixtures thereof,
characterized in that it has a total wax content of less than 7% by weight.

13. The composition according to claim 12, wherein the polyamide-type lipophilic polycondensate is chosen from ester-terminated polyamides, tertiary amide-terminated polyamides or polyalkyleneoxy-terminated polyamides or else ester-terminated poly(ester-amides).

14. The composition according to claim 13, wherein it comprises at least one ester-terminated polyamide obtained by condensation of a C36 diacid, on ethylene diamine, the remaining acid endings being esterified with cetyl alcohol, stearyl alcohol, or their mixture, and having the INCI name: ETHYLENEDIAMINE/STEARYL DIMER DILINOLEATE COPOLYMER.

15. The composition according to claim 13, which comprises at least one poly (ester-amide) with ester termination obtained by condensation of a C36 diacid, on ethylene diamine; the remaining acid endings being esterified with stearyl alcohol, and having the INCI name: POLYAMIDE-8.

16. The composition according to claim 12, wherein the lipophilic polyamide polycondensate is present in an active material content ranging from 5 to 30% by weight, by weight relative to the total weight of said composition.

17. The composition according to claim 12, which comprises:
a polyamide-type lipophilic polycondensate with the INCI name POLYAMIDE-8,
a compound promoting the adhesion of the composition to keratin materials chosen from the group consisting of:
vinylpyrrolidone (VP) and alkene copolymers having from 2 to 20 carbon atoms,
sugar esters,
and their mixture.

18. The composition according to claim 12, which comprises one or more hydrocarbon oils.

19. The composition according to claim 12, which further comprises an additional ingredient chosen from antioxidants, perfumes, preservatives, cosmetic active ingredients, fillers, and mixtures thereof.

20. The composition according to claim 12, which further comprises one or more UV filters.

21. The composition according to claim 12, which is a liquid composition for care and/or makeup for the skin and/or lips, or a solid composition or an anhydrous stick for care and/or makeup for the skin and/or lips.

22. The composition according to claim 12, which comprises in addition at least one coloring material.

23. The composition according to claim 16, which is a solid composition in which the lipophilic polyamide polycondensate is present in an active material content ranging from 15% to 30% by weight, relative to the total weight of said composition.

24. The composition according to claim 17, which comprises in addition at least one coloring material.

25. The composition according to claim 24, which comprises:
a polyamide-type lipophilic polycondensate with the INCI name POLYAMIDE-8, in a content ranging from 5 to 30% by weight, in particular from 15% to 30% by weight relative to the total weight of the composition,
a compound promoting the adhesion of the composition to keratin materials chosen from the group consisting of:
vinylpyrrolidone (VP) and alkene copolymers having from 2 to 20 carbon atoms,
sucrose acetate isobutyrate (SAIB),
and their mixture,
and in addition a coloring material.

26. A cosmetic method for the care and/or makeup of keratin materials comprising the application, to said keratin materials, of a composition according to claim 12.
